(19) United States
(12) Plant Patent Application Publication
(10) Pub. No.: US 2002/0002723 P1
(43) Pub. Date: Jan. 3, 2002

(54) Gladiolus plant named ' Princess Lee'

(76) Inventor: John O. Zipperer III, Ft. Myers, FL (US)

Correspondence Address: Laura G. Barrow, Esq., P.O. Box 215, Estero, FL, 33928-0215, US

(21) Appl. No.: 09/877,573

(22) Filed: Jun. 8, 2001

(30) Foreign Application Priority Data
Jun. 9, 2000 (EP) 2000/0893

(51) Int. Cl. A01H 5/00

(52) U.S. Cl. PLT/301

(57) ABSTRACT

A new and distinct gladiolus variety, designated ‘Princess Lee’ is shown and described. The variety is a true pink variety having straight, strong stems, a large bloom size, and good resistance to attack by insects and foliar diseases.

The present invention comprises a new and distinct variety of a Gladiolus plant referred to by the variety name ‘Princess Lee.’

### BRIEF DESCRIPTION OF THE PHOTOGRAPHS

FIGS. 1 and 2 are photographs of a ‘Princess Lee’ variety plant in bloom.

FIG. 3 is a photograph of the ‘Princess Lee’ variety plant prior to blooming.

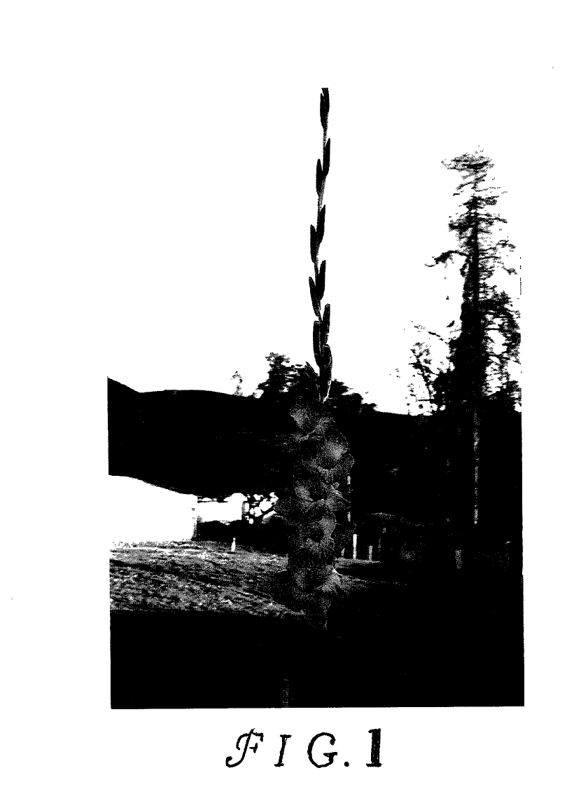

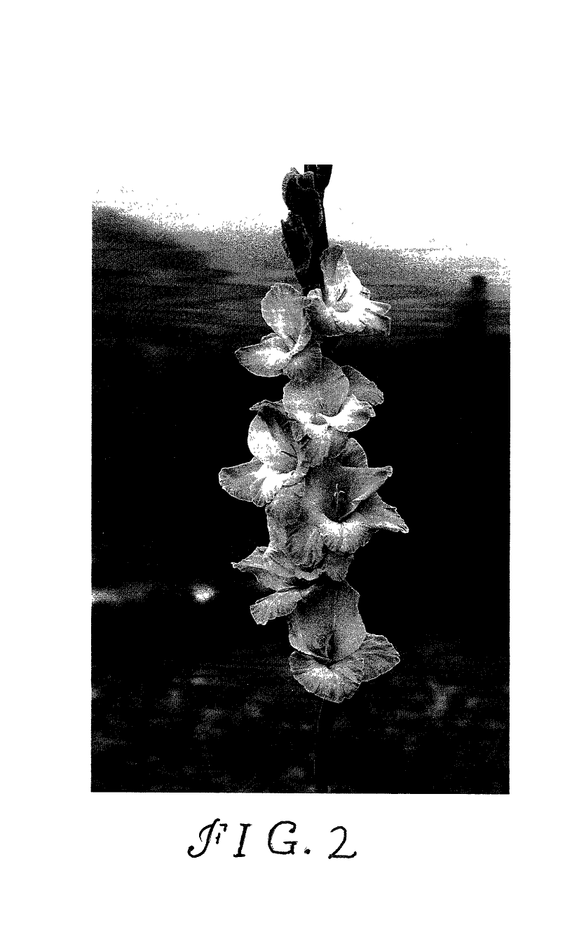

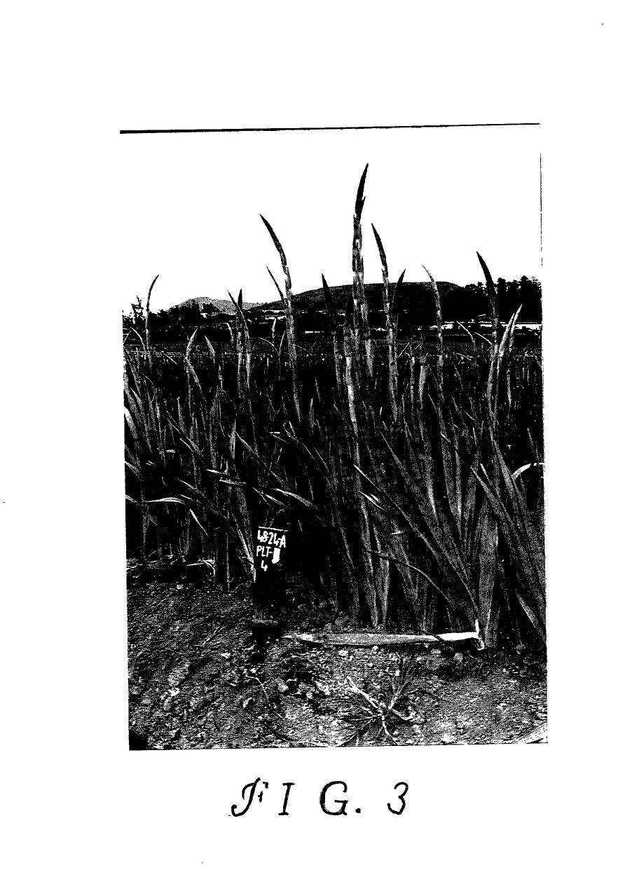

### ASEXUAL REPRODUCTION

The new variety was originated by the Applicant in a controlled proprietary breeding program in Ft. Myers, Fla. wherein selected gladiolus varieties were crossed. (The provisional breeder's designation “48-24”was used in a related application for a Community plant variety right filed earlier in the European Union Community Plant Variety Office). The female parent was a gladiolus variety named ‘Dr. Magee,’ characterized in part by having a small pink bloom, a short stem having a short flower head, and high resistance to Fusarium fungi species. The male parent was gladiolus variety named ‘White Prosperity,’ a short-headed, light-stemmed white variety that prefers long lengths. The seeds were planted in Ft. Myers, Fla., and the selection of ‘Princess Lee’ variety was made in Spring 1988. Asexual reproduction of the ‘Princess Lee’ variety was achieved by collecting cormels from the first corm. All subsequent asexual reproductions of the ‘Princess Lee’ variety remain true to the original variety type.

### BOTANICAL DESCRIPTION

The accompanying color photographs show the inflorescence and various stages of blooming of the ‘Princess Lee’ variety plant.

The bud size of the ‘Princess Lee’ variety is about 7 cm in length. The flowers of the variety each consist of a total of six petals. More specifically, the flower consists of three large petals oriented on the top of the bloom, two medium-size petals on the lower half of the bloom (positioned at 4:00 and 8:00), and a small-size petal positioned between the two medium-size petals on the lower half of the bloom. Alternatively, all three petals on the lower half of the bloom may be medium-sized. The diameter of the entire bloom is about 12 cm.

The color of the corolla bloom is a magenta deep within the throat of the bloom, followed by a true pink, then becoming an orange pink color near the edge of the petals with orange flecks on the margin of the petals.

The spike of the plant is about 123 cm and comprises 16 florets when grown from #1 size corms (1.5 inch) in Ecuador. The head length is about 56 cm. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes). The pistils of the flower are white and the stamens are pale yellow.

The corms of the ‘Princess Lee’ variety are typical for the gladiolus.

The leaf color of the ‘Princess Lee’ variety is green. The leaf size is about 65 cm (length) by 4 cm (width).

### GENERAL OBSERVATIONS

The ‘Princess Lee’ is a true pink gladiolus variety having very straight, strong stems, a large bloom size (i.e. 12 cm diameter), medium resistance to Fusarium and Curvalaria fungal species and other foliar diseases, and good resistance to attack by insects.

The ‘Princess Lee’ variety takes about 90 days to harvest in Ecuador, like the gladiolus variety ‘Advance.’ The variety is slow to open and should be harvested with three to four flowers showing color. The ‘Princess Lee’ variety can hold seven blooms open simultaneously in a vase of water after harvest from a tight cut stem.

Compared to the ‘Spic & Span’ variety, the ‘Princess Lee’ has a longer head length, is has a heavier stem, and has a longer spike length (about 10 cm longer).

I claim:

1. A new and distinct gladiolus variety ‘Princess Lee’, as shown and described herein.
